(19) United States
(12) Plant Patent Application Publication
(10) Pub. No.: US 2014/0173797 P1
(43) Pub. Date: Jun. 19, 2014

(54) Petunia plant named 'Archie's Lime on Yellow'

(50) ;

(71) Applicant: Elmer Lee Archibald, JR., Kernersville, NC (US)

(72) Inventor: Elmer Lee Archibald, JR., Kernersville, NC (US)

(21) Appl. No.: 13/694,641

(22) Filed: Dec. 19, 2012

(51) Int. Cl. A01H 5/00

(52) U.S. Cl. PLT/356.1

(57) ABSTRACT

A new and distinct flowering Petunia plant with a strong and long-lasting yellow flower color and good tolerance to hot weather conditions.

### BOTANICAL CLASSIFICATION

Petunia×hybrida

### VARIETAL DENOMINATION

‘Archie's Lime on Yellow’

### BACKGROUND OF THE INVENTION

The present invention comprises a new and distinct cultivar of petunia plant known by the varietal name ‘Archie's Lime on Yellow’. The new variety was discovered in April 2012 in a cultivated area as a naturally-occurring sport of many petunia varieties in a greenhouse in North Carolina. The new variety differs from petunia plant ‘Archie's Red2Purple’ (concurrently applied-for) in flower color and growth habit, however, the new variety is similar to the compared variety in its tolerance to hot weather conditions. The new variety has been found to retain its distinctive characteristics and remain true to type through successive propagations.

The following trait distinguishes ‘Archie's Lime on Yellow’ as a new and distinct cultivar:

1. Strong and long-lasting yellow flower color.

### DESCRIPTION OF THE DRAWINGS

The accompanying photographic drawings illustrate the new variety, with the color being as nearly true as is possible with color illustrations of this type:

FIG. 1 is a close-up photograph of a flower of the new variety;

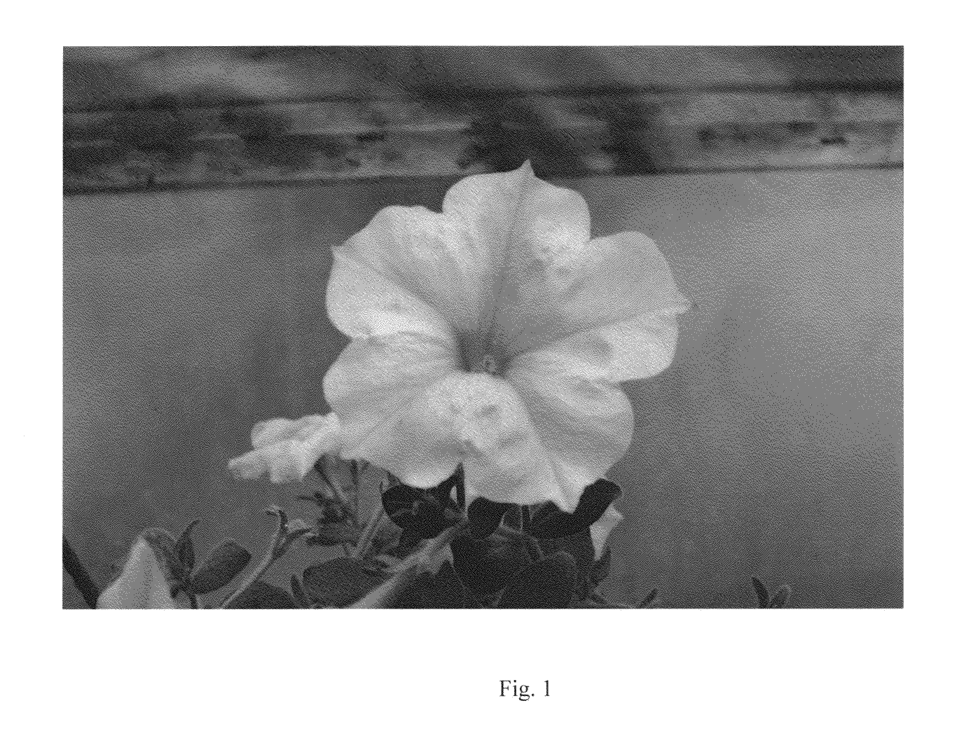

FIG. 2 is a close-up photograph of multiple flowers of the new variety; and

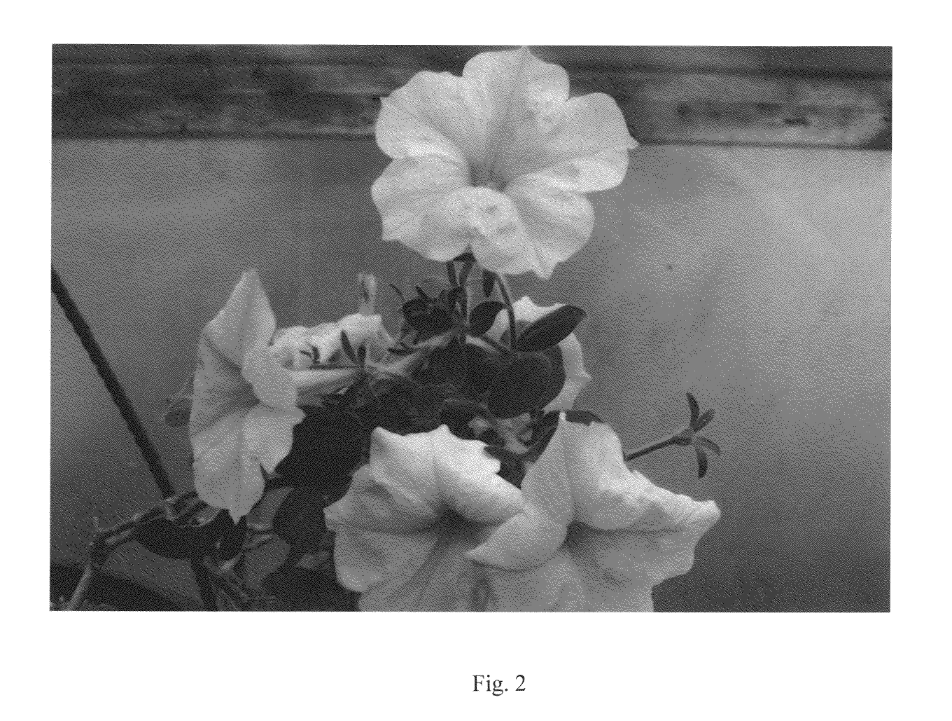

FIG. 3 is photograph of an entire plant of the new variety.

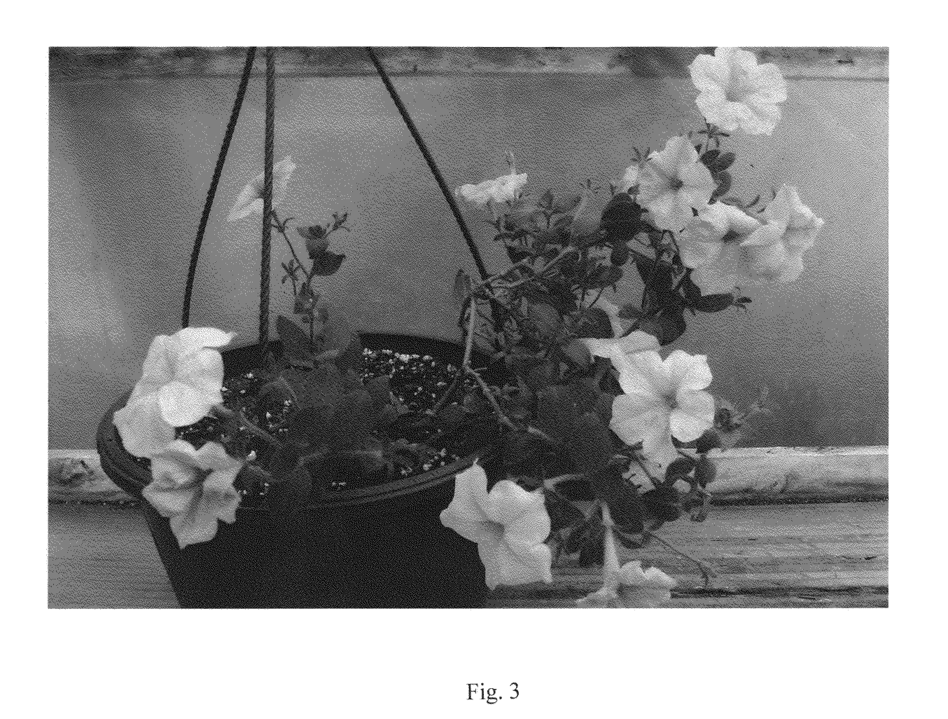

### DESCRIPTION OF THE PLANT

The following detailed description sets forth characteristics of the new cultivar. The data which defines these characteristics were collected by asexual reproductions by tip cuttings carried out in North Carolina in 2012. Plants for the description were 5 months old, grown in a greenhouse under 30% shade cloth in 10 inch hanging baskets. Average day temperatures were 70° F. and night temperatures were 50° F. The color readings were taken in a greenhouse under natural light. Color references are primarily to the 2001 R.H.S. Colour Chart of The Royal Horticultural Society of London.
Plant:
   Form.—Mounded.
   Height (from soil to top of flowers).—11 inches.
   Diameter.—17 inches.
   Vigor.—Medium.
   Branching habit.—Medium.
   Lateral branches.—Form: Mounded. Color: 1B to 1C. Texture: Rough. Length: 3-4 cm. Diameter: 2 inches. Internode length: 1.5 cm. Quantity: Numerous.
   Foliage.—Arrangement: Single. Number of leaves per branch: Varies by branch length; a 9 inch branch has 40 leaves. Size of leaf: Length: From 2.5-3 cm. Width: From 1.7-2 cm. Shape of leaf: Ovate. Shape of apex: Pointed. Shape of base: Rounded. Texture: Rough. Margin: Entire. Color: Young leaves: Upper surface: 137C. Lower surface: 138B. Mature leaves: Upper surface: 137C. Lower surface: 137D. Petiole: Length: 1.5 cm. Diameter: 0.3 cm. Color: 137C. Veins: Number: 5. Color: Upper surface: 150A. Lower surface: 150B.
Flower:
   Natural flowering season.—April through October in North Carolina (Zone 8A).
   Flower type and habit.—Single; upward; funnel-shaped; 5-petals.
   Number of flowers per plant.—A small plant has approximately 20.
   Corolla.—Diameter: 5.5 cm. Flower tube length: 2.9 cm. Flower tube diameter at distal end: 0.2 cm. Flower tube diameter at proximal end: 0.5 cm. Petal length from throat: 2 cm. Petal width: 2.4 cm. Petal quantity: 5 parts fused at base. Petal apex shape: Circular. Texture: Reflective. Color: When opening: Upper surface: 1B. Lower surface: 1B. Fully opened: Upper surface: 1C. Lower surface: 1C. Flower throat (inside): 1C. Flower throat, vein: 150A. Flower tube (outside): 150C. Flower tube, vein: 150A. Petal color, fading to: 154D.
   Peduncle.—Length: 3 cm. Diameter: 0.2-0.3 cm. Color: 150A. Orientation: Angled. Strength: Sturdy.
   Bud.—Shape: Trumpet. Length: 3.1 cm. Diameter: 0.9 cm. Color: 149B.
   Calyx.—Shape: Cup. Length: 3.8 cm. Diameter: 0.6 cm. Sepal shape: Cup. Sepal texture: Rough. Sepal color: Upper surface: 144A. Lower surface: 144B.
   Disease resistance.—No resistance to diseases noted to date.
   Fragrance.—None.
   Weather tolerance.—Good tolerance to hot weather.
   Lasting quality.—Good.
   Seed/fruit production.—None observed.
Reproductive organs:
   Stamens.—Number: 1. Filament length: 1.6 cm. Anthers: Shape: Capsule. Length: 1 cm. Color: Yellow. Pollen: Color: White. Amount: Average.
   Pistil.—Length: 1.1 cm. Style: Length: 1 cm. Color: Green. Stigma: Shape: Round. Color: Green. Ovary color: White.

I claim:

1. A new and distinct variety of Petunia plant substantially as shown and described.
